(19) United States
(12) Patent Application Publication
(10) Pub. No.: US 2022/0228202 A1
(43) Pub. Date: Jul. 21, 2022

(54) POSITIVE PRESSURE DRIVEN FLOW FOR MULTIPLEXED FLUORESCENCE IN SITU HYBRIDIZATION IMAGING SYSTEM

(71) Applicant: Applied Materials, Inc., Santa Clara, CA (US)

(72) Inventor: Tze Howe Charn, Singapore (SG)

(21) Appl. No.: 17/153,791

(22) Filed: Jan. 20, 2021

(51) Int. Cl. C12Q 1/6841; B01L 3/00; G01N 21/64

(52) U.S. Cl. CPC C12Q 1/6841; B01L 3/527; B01L 3/567; G01N 21/6458; G01N 21/6486; B01L 2400/0694; B01L 2200/0684; B01L 2200/0621; B01L 2400/0487; B01L 2200/0689; B01L 2300/14; B01L 2300/048; B01L 2400/0666

(57) ABSTRACT

A fluorescent in-situ hybridization imaging system, including a flow cell to contain a sample to be exposed to fluorescent probes in a reagent; a plurality of reagent reservoirs, each reagent reservoir including a container to hold a liquid reagent; a valve system to control flow from one of a plurality of reagent reservoirs to the flow cell; a pressure source coupled to each of the plurality of reagent reservoirs to apply a positive pressure to liquid reagent in the container and urge the liquid reagent to flow toward the flow cell; and a fluorescence microscope including a variable frequency excitation light source and a camera positioned to receive fluorescently emitted light from the sample.

### TECHNICAL FIELD

This specification relates to a driving flow through a flow cell in a fluorescence in-situ hybridization imaging system.

### BACKGROUND

It is of great interest to the biotech community and pharmaceutical industry to develop methods for visualizing and quantifying multiple biological analytes—e.g., DNA, RNA, and protein—within a biological sample—e.g., tissue resection, biopsy, cells grown in culture. Scientists use such methods to diagnose/monitor disease, validate biomarkers, and investigate treatment. To date, exemplary methods include multiplex imaging of antibodies or oligonucleotides (e.g., RNA or DNA) labeled with a functional domain to a biological sample.

Multiplexed fluorescence in-situ hybridization (mFISH) imaging is a powerful technique to determine gene expression in spatial transcriptomics. In brief, a sample is exposed to multiple oligonucleotide probes that target RNA of interest. These probes have different labeling schemes that will allow one to distinguish different RNA species when the complementary, fluorescent labeled probes are introduced to the sample. Then the sequential rounds of fluorescence images are acquired with exposure to excitation light of different wavelengths.

Exposing the sample generally includes flowing one or more solutions through a flow cell containing a sample. Traditional methods include the use of downstream rotary peristaltic pumps to generate negative pressure and draw reagents into and through the flow cell.

### SUMMARY

In one aspect, a fluorescent in-situ hybridization imaging system, including a flow cell to contain a sample to be exposed to fluorescent probes in a reagent; a plurality of reagent reservoirs, each reagent reservoir including a container to hold a liquid reagent; a valve system to control flow from one of a plurality of reagent reservoirs to the flow cell; a pressure source coupled to each of the plurality of reagent reservoirs to apply a positive pressure to liquid reagent in the container and urge the liquid reagent to flow toward the flow cell; and a fluorescence microscope including a variable frequency excitation light source and a camera positioned to receive fluorescently emitted light from the sample.

In some embodiments, each reagent reservoir of the plurality of reagent reservoirs further includes a sealing lid, including an inlet configured to receive pressurized gas from the pressure source above an upper surface of the liquid reagent, and an outlet configured to permit flow of the liquid reagent out of the reagent reservoir from below the upper surface of the liquid reagent. The inlet is further configured to detachably connect to the sealing lid, and the outlet is further configured to detachably connect to the sealing lid. The pressure source includes a gas pump for delivering pressurized gas to and a pressure regulator for regulating the pressurized gas. The valve system includes a plurality of valves, each valve of the plurality of valves corresponding to one reagent reservoir of the plurality of reagent reservoirs.

The system further includes a first solenoid valve (e.g., 3/2 valve) in fluid communication between the valve system and the flow cell. The system further includes a second solenoid valve in fluid communication with the flow cell. The system further includes a controller to control the valve system, configured to synchronize movement of the valve system such that only one liquid reagent flows to the flow cell at a time. The system further includes a manifold, the manifold including a plurality of outlet ports, each outlet port being in fluid communication with one reagent reservoir. The liquid reagent includes oligonucleotide probes. The liquid reagent includes a buffer. The liquid reagent includes a purge fluid, an imaging buffer, or a bleach buffer.

In another aspect, a method for using positive pressure in a fluorescent in-situ hybridization imaging system, the method including applying a positive pressure to a plurality of liquid reagents in a plurality of reagent reservoirs; selecting a first liquid reagent from the plurality of liquid reagents; supplying the first liquid reagent as driven by the positive pressure to a flow cell containing a sample; and obtaining a first fluorescence microscope image of the sample.

In some embodiments, the first liquid reagent includes oligonucleotide probes having a first nucleotide sequence. The method further includes selecting a second liquid reagent from the plurality of liquid reagents; and supplying the second liquid reagent as driven by the positive pressure to a flow cell containing a sample. The second liquid reagent includes a purge fluid or a photobleaching buffer. The method further includes selecting a third liquid reagent from the plurality of liquid reagents; supplying the third liquid reagent as driven by the positive pressure to a flow cell containing a sample; and obtaining a second fluorescence microscope image of the sample. The third reagent includes oligonucleotide probes having a third nucleotide sequence. The second liquid reagent includes a purge fluid, a washing bugger, an imaging buffer, or a photobleaching buffer. Applying the positive pressure includes applying gas pressure to the plurality of liquid reagents. Advantages of implementations can include, but are not limited to, one or more of the following.

Using positive pressure fluid displacement systems in conjunction with a flow cell for fluorescence in-situ hybridization imaging systems can prevent a number of standing issues. Positive pressure displacement systems distribute gas pressure equally to downstream components. Gas lines downstream of the pressure regulator can be of flexible length and design, allowing for additional regulating manifolds and the capability of multiple flow cells for imaging samples.

Constant pressure applied to headspace within reagent tubes allows for constant flow rates downstream in the liquid circuitry. As the liquid reagent travels to the flow cell, constant pressure reduces turbulent flow as the reagent is introduced to the sample. The reduced turbulence increases the likelihood of the sample maintaining position within the flow cell, and reduces the need for the imaging system or technician to reacquire a new position or z-height of the sample, thereby yielding higher image acquisition rates and reducing system reagent use by reducing the number of failed FOV images.

Positive pressure displacement of liquids also produces smoother flow at low flow rates. This is particularly useful for sensitive or small samples that could be driven from their spatial positions by higher flow rates through the flow cells. Because the positive pressure equally distributes across the available headspaces in near-real time, multiple reagents can be delivered to the sample simultaneously, rather than sequentially.

The details of one or more embodiments are set forth in the accompanying drawings and the description below. Other features, aspects, and advantages will become apparent from the description, the drawings, and the claims.

### BRIEF DESCRIPTION OF THE DRAWINGS

FIG. 1 is a schematic diagram of an apparatus for multiplexed fluorescence in-situ hybridization imaging using positive pressure displacement.

FIG. 2 is a schematic diagram of a positive pressure displacement system including a flow cell.

FIG. 3 is a schematic cross-sectional diagram of a positive pressure displacement system displacing reagents from a tube.

FIG. 4 is a schematic diagram of a positive pressure displacement system including two flow cells.

FIG. 5 is a flow chart of a method of mFISH imaging.

Like reference numbers and designations in the various drawings indicate like elements.

### DETAILED DESCRIPTION

Multiplex fluorescent in-situ hybridization imaging (mFISH) systems generally include a peristaltic pump downstream of the imaging flow cell to produce negative pressures, thereby drawing liquids from the flow cell and upstream components and driving waste into a disposal container. However, negative pressure flow cells tend to generate turbulent flow of liquids, e.g., reagents, buffs, or washes, through the flow cell. This can cause turbulent motion of the sample within the flow cell, changing the position and elevation of the sample while being imaged.

In particular, peristaltic pumps can generate an uneven flow rate, termed pulsation. This pulsation causes turbulent flow through sensitive components, such as flow cells. Pulsation through a fluidic circuit can be affected by line length and flow rate.

The pulsation of peristaltic flow can also cause imaging issues for a sensitive imaging system capable of changes in focal plane and FOV lateral resolution measured in microns. The uneven flow through the flow cell can cause sensitive or small samples to be moved from their previously imaged position, resulting in reacquiring images at different focal planes, making registration between images taken at different times more difficult, or loss of imaging data.

The use of negative pressure downstream of a flow cell also requires that fluid tube lengths be highly uniform to maintain the pressure from the peristaltic pump, through the flow cell, the selector, reagent manifold, and individual reagent tubes. Negative pressure systems can also induce cavitation, e.g., bubble formation, in solutions with large volumes of dissolved gases in fluids being transported through the liquid circuitry. Entrained bubbles in the fluid circuitry also lead to imaging problems if the bubbles travel through the flow cell in between rounds of imaging.

In contrast, a positive pressure can provide more precise control over pressure at each gas and liquid junction in an mFISH imaging system. Because the gas pressure can be evenly distributed from the gas manifold, the flow rate from each reagent tube can be more finely controlled without pressure fluctuations that peristaltic pumps introduce. The flexibility of positive gas pressure distribution also allows for simplified addition of further flow cells and necessary components.

Changes in gas pressure can occur at shorter time scales than liquid pressure, and the headspace in reagent tubes can provide a buffer, reducing pressure fluctuations in the downstream liquid components. This reduces turbulence in the flow cell and reduces the chance a sample can be moved from the imaging position.

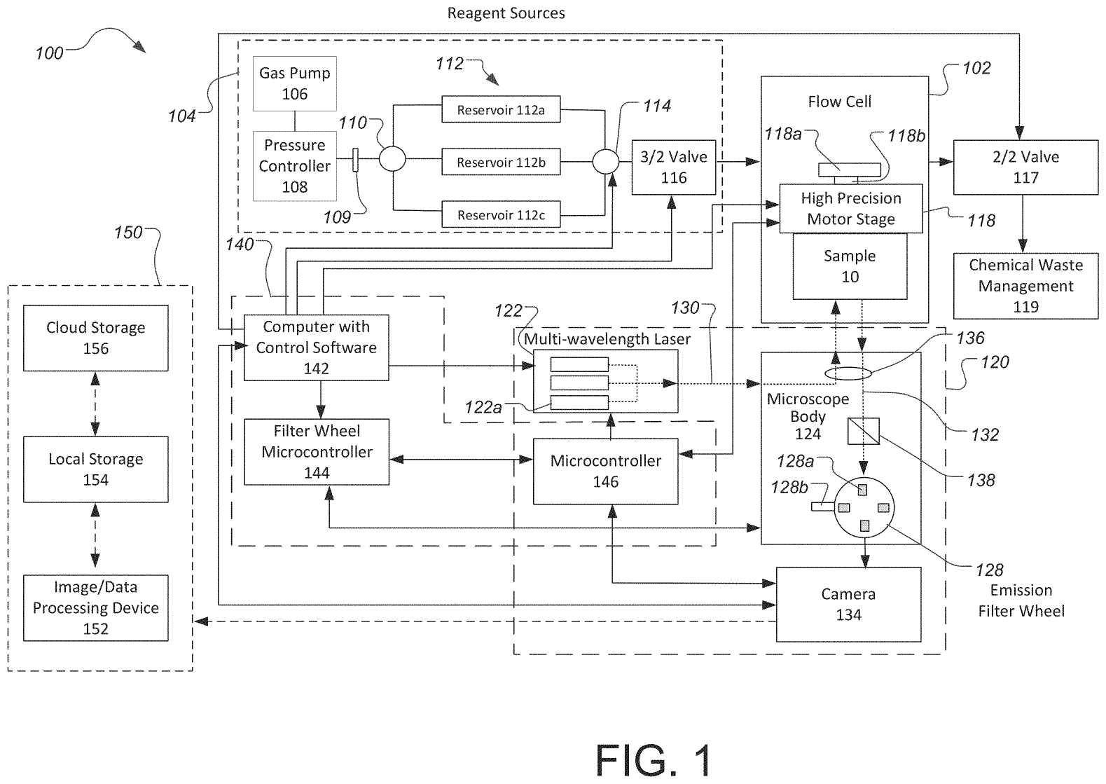

Referring to FIG. 1, a multiplexed fluorescent in-situ hybridization (mFISH) imaging system 100 includes a flow cell 102 to hold a sample 10, a fluorescence microscope 120 to obtain images of the sample 10, and a control system 140 to control operation of the various components of the mFISH imaging system 100. The control system 140 can include a computer 142, e.g., having a memory, processor, etc., that executes control software.

The fluorescence microscope 120 includes an excitation light source 122 that can generate excitation light 130 of multiple different wavelengths. In particular, the excitation light source 122 can generate narrow-bandwidth light beams having different wavelengths at different times. For example, the excitation light source 122 can be provided by a multi-wavelength continuous wave laser system, e.g., multiple laser modules 122a that can be independently activated to generate laser beams of different wavelengths. Output from the laser modules 122a can be multiplexed into a common light beam path.

The fluorescence microscope 120 includes a microscope body 124 that includes the various optical components to direct the excitation light from the light source 122 to the flow cell 102. For example, excitation light from the light source 122 can be coupled into a multimode fiber, refocused and expanded by a set of lenses, then directed into the sample by a core imaging component, such as a high numerical aperture (NA) objective lens 136. When the excitation channel needs to be switched, one of the multiple laser modules 122a can be deactivated and another laser module 122a can be activated, with synchronization among the devices accomplished by one or more microcontrollers 144, 146.

The objective lens 136, or the entire microscope body 124, can be installed on a vertically movable mount coupled to a Z-drive actuator. Adjustment of the Z-position, e.g., by a microcontroller 146 controlling the Z-drive actuator, can enable fine tuning of focal position. Alternatively, or in addition, the flow cell 102 (or a stage 118 supporting the sample in the flow cell 102) could be vertically movable by a Z-drive actuator 118b, e.g., an axial piezo stage. Such a piezo stage can permit precise and swift multi-plane image acquisition.

The sample 10 to be imaged is positioned in the flow cell 102. The flow cell 102 can be a chamber with cross-sectional area (parallel to the object or image plane of the microscope) with an area of about 2 cm by 2 cm. The sample 10 can be supported on a stage 118 within the flow cell, and the stage (or the entire flow cell) can be laterally movable, e.g., by a pair of linear actuators 118a to permit XY motion. This permits acquisition of images of the sample 10 in different laterally offset fields of view (FOVs). Alternatively, the microscope body 124 could be carried on a laterally movable stage.

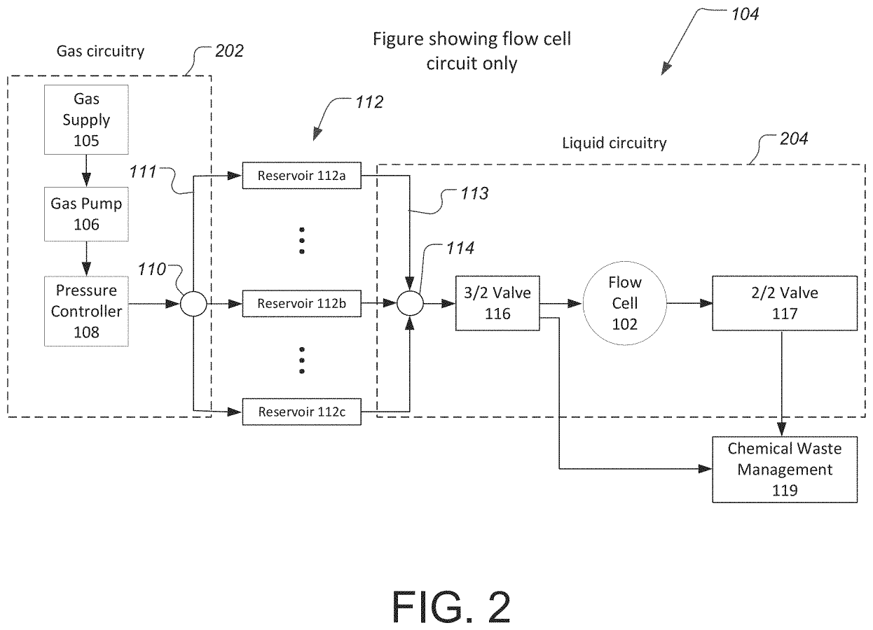

A positive pressure reagent delivery system 104 connects to an entrance to the flow cell 102. Broadly, the reagent delivery system 104 includes a pressure management system, directing pressurized gas to the headspace of a number of reservoirs 112, at least some of which are reagent reservoirs. The pressurized gas drives liquid, e.g., a liquid reagent, e.g., from reservoir 112a, to a valve system 114 and further to the flow cell 102. The positive pressure reagent delivery system 104 and included components are explained in more detail with regard to FIG. 2.

FIG. 2 shows an example positive pressure reagent delivery system 104 including gas flow subsystem 202 to create and distribute the gas pressure to the headspace of the reservoirs 112, and liquid flow subsystem 204 for supplying selected liquids to the flow cell 102 and directing spent solutions to the chemical waste management system 119.

The gas flow subsystem 202 of the reagent delivery system 104 includes a pressure source for supplying gas pressure to the downstream components. FIG. 2 depicts the pressure source as a gas pump 106 in fluid communication with a gas supply 105, e.g., and a pressure controller 108. The gas supply 105 can be a source of purified gas, e.g., substantially particle free gas, such as a tank or an industrial gas supply line, or the gas supply can simply be surrounding atmosphere. The gas can be air, nitrogen, argon, or other molecular inert gas or mixture thereof.

The gas pump 106 is a pneumatic pump that is compatible with the imaging environment. Because the pressurized gas will be in contact with sensitive reagent solutions, a pump that generates relatively little contamination, such as an oil-free compressor pump, can be used as the gas pump 106. The gas pump 106 intakes gas from the gas supply 105, compresses the gas, and supplies gas (e.g., air) compressed to above local atmospheric pressure. For example, the gas supplied to the gas circuitry can be 1 psi above local atmospheric pressure or more (e.g., 1 psi, 1.5 psi, 2 psi, 2.5 psi or more). The pressure controller 108 intakes the compressed gas and outputs the gas at a regulated pressure to the manifold 110. Examples of pressure controller 108 include a pressure reducing regulator (e.g., reduces the input pressure of the gas to a desired value at its output) or a back-pressure regulator (e.g., maintains the set pressure at its inlet side by opening to allow flow when the input pressure exceeds the set value). In some implementations, the pressure source can be a container (e.g., a tank) of compressed gas.

In some implementations, the compressed gas can be filtered by a filter 109, for example, to remove particulates, aerosolized liquids, or biological agents to prevent contamination of the liquid reagents in the reservoirs 112. The filtration can occur before being compressed, between the gas pump 106 and the pressure controller 108, or following the pressure controller 108.

The pressure controller 108 outputs the compressed gas at a regulated pressure to a gas manifold 110. The gas manifold 110 includes an inlet port to accept the pressurized gas, and fluidly connects the inlet port to one or more outlet ports, e.g., multiple outlet ports, thereby equally distributing the compressed gas to each outlet port. For example, if pressure controller 108 supplies compressed gas at 1 psi to the input of manifold 110, each output will supply compressed gas at 1 psi to connected components.

Each outlet port of the manifold 110 is in fluid connection with the headspace of one corresponding reservoir 112 through a gas line 111. In general, the gas manifold 110 includes at least as many outlet ports as there are reservoirs 112. In some implementations, gas manifold 110 can have 5 or more outlet ports (e.g., 8 or more, 10 or more, 12 or more, 15 or more, or 20 or more).

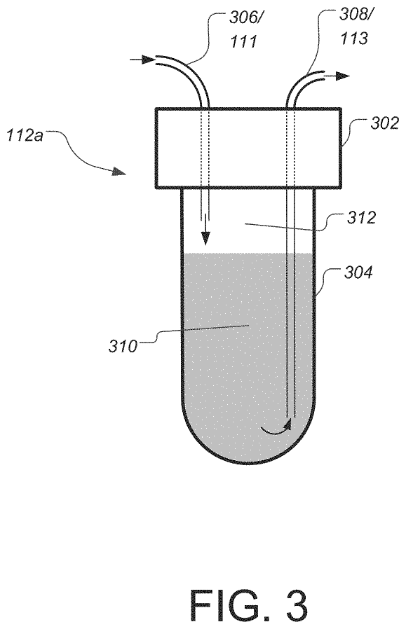

FIG. 3 shows an example reservoir 112a and the corresponding connections to the gas flow subsystem 202 and liquid flow subsystem 204. The reservoir 112a includes a lid 302, a vessel 304, a first conduit 306 (which provides the gas line 111), and a second conduit 308 (which provides a liquid line 113). The reagent vessel 304 is a receptacle for containing an amount of liquid 310, e.g., a reagent. The remaining volume of the vessel 304 above the surface of the liquid 310 is termed “headspace” 312. The first conduit 306 has an opening positioned to be in the headspace 312 during operation of the reagent delivery system 104, i.e., when the reservoir is partially filled with liquid. In contrast, the second conduit 308 has an opening positioned to be below the surface of liquid 310 during operation. Although the implementation in FIG. 3 illustrates the first and second conduits 306, 308 extending through the lid 302, the conduits could instead open directly into sidewalls of the vessel 304.

The first conduit 306 can be provided by a material capable of maintaining the regulated gas pressure from the manifold 110, e.g., flexible or rigid plastic tubing, glass or metal piping, etc. Flexible materials such as plastic, rubber, or silicone provide possible advantages in reducing pressure fluctuations in the gas being distributed. The second conduit 308 can be composed of similar materials to the first conduit 306. Examples of second conduits 308 include high pressure liquid chromatography tubing composed of ethylene tetrafluoroethylene (ETFE) or poly ether ether ketone (PEEK). The first and second conduits 306, 308 can have equal cross-sectional area, or have different cross-sectional areas.

An amount of liquid can be delivered into the vessel 304, e.g., manually by an operator or automatically through another unillustrated delivery line, so that the headspace includes 5% or more of the total interior volume of the reservoir (e.g., 5% or more, 10% or more, 20% or more, or 30% or more). The example vessel 304 of FIG. 3 is cylindrical with a hemispherical bottom (e.g., a test tube), though in some implementations, the vessel 304 can be a different shape (e.g., a bottle). Examples of the vessel 304 can include glass or polymer vessels, and the vessel 304 can have an interior volume sufficient to contain 1 mL or more (e.g., 2 mL or more, 5 mL or more, 10 mL or more, 20 mL or more, or 50 mL or more).

The reservoir lid 302 includes an upper surface, a side surface extending from the rim of the upper surface, and two ports on the upper surface. The reservoir lid 302 detachably secures to the opening of vessel 304 to form a gas-tight seal (e.g., a sealing lid). The ports can be cylindrical in shape and provide access from the outer surface of the seal to the interior volume of the reagent vessel 304 when the reservoir lid 302 is secured to the vessel 304. In some implementations, the ports include gaskets to form gas-tight seals with objects traversing the port.

The first conduit 306 delivers pressurized gas from the manifold 110 to the headspace 312 of the reservoir 112a, thereby increasing the gas pressure within the vessel 304. The increased gas pressure of the headspace 312 exerts a downward force equally distributed across the upper surface of the liquid 310.

At least one reservoir holds a liquid reagent. In particular, different reservoirs can hold different reagents. Each different liquid reagent includes a different set of one or more different types of oligonucleotide readout probes. Each different type of readout probe targets a different nucleotide sequence on a different encoding probe (and thus targets a different RNA sequence), and/or has a different set of one or more fluorescent materials, e.g., phosphors, that are excited by different combinations of wavelengths.

One or more of the reservoirs 112 can contain other liquids for delivery to the flow cell 102, such as a purge fluid (e.g., DI water), or a buffer (e.g., a wash buffer, an imaging buffer, or a bleaching buffer), instead of a reagent.

The second conduit 308 extends below the upper surface of the liquid 310, for example, near to the bottom of the vessel 304. The increased gas pressure of the headspace 312 drives the liquid 310 into the open end of second conduit 308 and through second conduit 308 to downstream liquid flow subsystem 204.

Referring again to FIG. 2, the pressure controller 108 maintains the gas pressure within the manifold 110 which distributes the compressed gas to the headspace 312 of each connected reservoir 112. The liquid flow subsystem 204 of the reagent delivery system 104 includes the liquid lines 113 through which liquid 310 is driven from the reservoirs 112, and a valve system 114 for selecting one or more liquids to deliver to the flow cell 102. The liquid flow subsystem 204 can also include a first valve 116, e.g., a solenoid valve, to control flow of the liquid into the flow cell 102, and a second valve 117, e.g., a solenoid valve, to control flow of liquid out of the flow cell 102.

Liquid 310 from reservoirs 112 is driven through the second liquid lines 113, which terminate at the valve system 114. The valve system 114 is a selector valve, e.g., a rotary valve, including at least as many input ports as reservoirs 112 of the reagent delivery system 104, and a single output port. Each second conduit 308 extending from a corresponding reservoir 112 is in fluid connection with a single input port of the valve system 114. The valve system 114 connects a single input port corresponding to a single reservoir 112 to the output port, supplying a single liquid 310 to downstream components. The control system 140 controls the valve system 114 to switch between reservoirs 112 thereby selecting which liquid 310 is supplied to the solenoid valve 116.

The first valve 116 can be a selector valve with one inlet port and two outlet ports, e.g., a 3/2 valve. The first valve 116 operates to fluidly connect the inlet port with a single outlet port. The control system 140 controls which outlet port is connected. When the inlet port is in fluid connection with a first outlet port, there is no fluid connection to the second outlet port, and vice versa. One outlet is in fluid connection with the flow cell 102 and the other outlet port is in fluid connection with the chemical waste management system 119. When the first valve 116 connects the valve system 114 to the flow cell 102, the selected liquid 310 can flow to the flow cell 102. Any volume of liquid 310 remaining in the first valve 116 can be purged when the flow cell 102 is connected to the chemical waste management system 119. This additionally can prevent backflow from flow cell 102 entering the first valve 116.

The flow cell 102 receives the liquid 310 supplied from the first valve 116. The flow cell 102 also has an outlet in fluid connection with a second valve 117, e.g., a 2/2 valve, also controlled by the control system 140 to control flow of liquid, e.g., the reagent or purge fluid, through the flow cell 102. When the valves 116, 117 are operated by the control system 140 in unison, liquid flows from first valve 116, through the flow cell 102, and through the solenoid valve 117. In this manner, a selected liquid 310 is supplied to the flow cell 102 and used solution is displaced and directed to the chemical waste management system 119. In contrast, both valves 116, 117 can be closed to seal the flow cell 102.

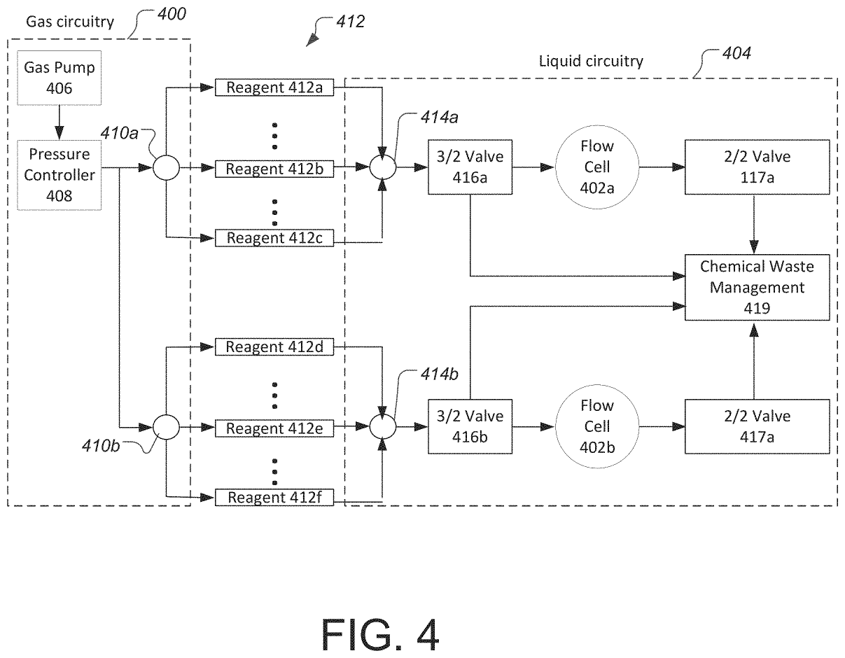

Referring now to FIG. 4, in some implementations, the reagent delivery system 104 can provide liquid 310 to multiple flow cells 402. The example system of FIG. 4 shows gas circuitry 400 including an gas pump 406 in fluid connection with a pressure controller 408 fluidly connected to two manifold 410a, 410b. In some implementations, the pressure controller 408 delivers equal gas pressure to manifolds 410a, 410b.

The manifolds 410a, 410b connect to a number of reservoirs 412, with manifold 410a connecting to the headspace 312 of reservoirs 412a-c and manifold 410b connecting to the headspace 312 of reservoirs 412d-f.

Valve system 414a receives liquid 310a-c from reservoirs 412a-c, whereas valve system 414b receives liquid 310d-f from reservoirs 412d-f. Each valve system 414a, 414b is controlled by control system 140 to deliver one liquid 310 to respective first solenoid valves 416a, 416b. As described above, the first solenoid valves 416a, 416b direct, upon control by the control system 140, the selected liquid 310 to the respective flow cell 402a, 402b or to the chemical waste management system 419. Assuming liquid is directed to a flow cell, liquid that is displaced from the flow cells 402a, 402b is driven to the second solenoid valves 417a, 417b, and on to the chemical waste management system 419.

Returning to FIG. 1, in operation, the control system 140 causes the light source 122 to emit the excitation light 130, which causes emission of light from fluorophores in the sample 10, e.g., of fluorophores in the probes that are bound to RNA in the sample and that are excited by the wavelength of the excitation light. The emitted light 132, as well as back propagating excitation light, e.g., excitation light scattered from the sample, stage, etc., are collected by an objective lens 136 of the microscope body 124.

The collected light can be filtered by a multi-band dichroic mirror 138 in the microscope body 124 to separate the emitted fluorescent light from the back propagating illumination light, and the emitted light is passed to a camera 134. The multi-band dichroic mirror 138 can include a pass band for each emission wavelength expected from the probes under the variety of excitation wavelengths. Use of a single multi-band dichroic mirror (as compared to multiple dichroic mirrors or a movable dichroic mirror) can provide improved system stability.

When triggered by a signal, e.g., from a microcontroller, image data from the camera can be captured, e.g., sent to an image processing system 150. Thus, the camera 134 can collect a sequence of images from the sample. The sequence of images can provide the visualization and can be analyzed to quantify a biological analyte, e.g., the expression of a gene.

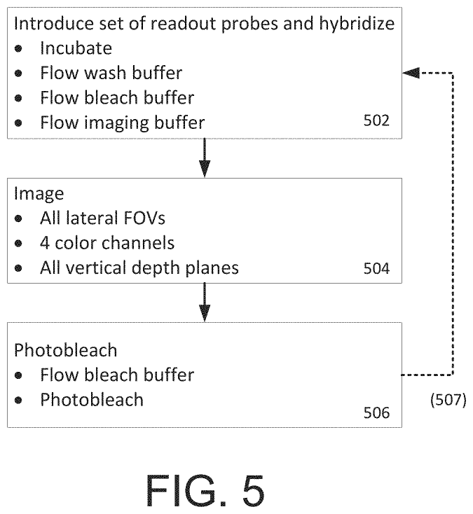

To provide context, the conventional mFISH traditional round of mFISH imaging and genetic identification relies on a series of nested steps including hybridization, imaging, and photobleaching. FIG. 5 demonstrates the workflow for a round of mFISH. Prior to mFISH imaging, encoding probes are added to a biological sample containing sequences to be targeted, e.g., by flowing the reagent from a selected reagent reservoir to the flow cell. The target nucleotide sequences are bound with a library of encoding probes, each encoding probe containing an encoding sequence that binds to a specific targeting sequence, and a hybridization region at each end of the encoding sequence. The hybridization regions are designed to bind the targeting sequences present in the set of readout probes, but not to the sequences of the sample.

The first round of hybridization of the readout probes (502) begins with the valve system 114 supplying a reagent containing readout probes to the flow cell 102 containing the sample 10. As described above, each readout probe includes a fluorophore coupled to an oligonucleotide targeting sequence designed to bind to one of the hybridization regions of the encoding probes. In practice, there can be multiple groups of readout probes, with readout probes within a group having the same oligonucleotide targeting sequence and the same fluorophore, but readout probes of different groups having oligonucleotide targeting sequences and different fluorophores that emit at different wavelengths. The total number of groups of readout probes can be equal to or less than the number of color channels the system is capable of imaging. For example, a control system 140 with a light source 122 with four laser modules can excite a set of four unique fluorophores in a sequence of four rounds of excitation and imaging.

The system performs an incubation step allowing the set of readout probes to penetrate the sample and hybridize with the encoding probes. The system then supplies one or more buffers from one or more selected reservoirs to the flow cell 102 via the valve system 114 to prepare the sample for imaging. The buffers can include a wash buffer which can include reagents to displace unbound and excessive components which may interfere with the assay, such as an astringent reagent (e.g., formamide). The buffers can further include a hybridization buffer to control stringency and eliminate residual fluorescent material or autofluorescence of the sample. The buffers can further include an imaging buffer to prepare the sample and probes for imaging, such as performing oxygen scavenging (e.g., glucose oxidase).

The system then performs an imaging step (504). The light source 122 consecutively excites the fluorophores of the set of readout probes localized within a selected FOV while the filter wheel 128 allows for the collection of the emitted fluorescence to form a fluorescent image. In some implementations, the imaging system may be configured, e.g., with a color camera, to image multiple fluorophores of different emission wavelengths simultaneously. This captures the lateral and vertical position of the readout probes hybridized in the preceding step.

The fluorophore of the hybridized readout probes are then photobleached (506). This begins by the valve system 114 supplying a volume of bleaching buffer to the flow cell 102 to displace and purge the imaging buffer. The photobleaching includes bathing the sample 10 in the flow cell 102 with high intensity light to photochemically render the readout probes hybridized within the sample fluorophores permanently unable to fluoresce. The light source is chosen to correspond to the fluorophores used in combination with the readout probes.

The process can then be repeated (507) with additional rounds of hybridization, buffer washes, imaging, and photobleaching. For example, an mFISH experiment can include between 4 and 20 rounds of hybridization and mFISH imaging with unique readout probes used in each round. Each round of hybridization can use a reagent from a different reservoir.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A fluorescent in-situ hybridization imaging system, comprising:
a flow cell to contain a sample to be exposed to fluorescent probes in a reagent;
a plurality of reagent reservoirs, each reagent reservoir including a container to hold a liquid reagent;
a valve system to control flow from one of a plurality of reagent reservoirs to the flow cell;
a pressure source coupled to each of the plurality of reagent reservoirs to apply a positive pressure to liquid reagent in the container and urge the liquid reagent to flow toward the flow cell; and
a fluorescence microscope including a variable frequency excitation light source and a camera positioned to receive fluorescently emitted light from the sample.

2. The system of claim 1, wherein each reagent reservoir of the plurality of reagent reservoirs further comprises a sealing lid, comprising an inlet configured to receive pressurized gas from the pressure source above an upper surface of the liquid reagent, and an outlet configured to permit flow of the liquid reagent out of the reagent reservoir from below the upper surface of the liquid reagent.

3. The system of claim 2, wherein the inlet is further configured to detachably connect to the sealing lid, and the outlet is further configured to detachably connect to the sealing lid.

4. The system of claim 1, wherein the pressure source comprises a gas pump for delivering pressurized gas to and a pressure regulator for regulating the pressurized gas.

5. The system of claim 1, wherein the valve system comprises a plurality of valves, each valve of the plurality of valves corresponding to one reagent reservoir of the plurality of reagent reservoirs.

6. The system of claim 1, further comprising a first solenoid valve (e.g., 3/2 valve) in fluid communication between the valve system and the flow cell.

7. The system of claim 6, further comprising a second solenoid valve in fluid communication with the flow cell.

8. The system of claim 7, further comprising a controller to control the valve system, configured to synchronize movement of the valve system such that only one liquid reagent flows to the flow cell at a time.

9. The system of claim 1, the system further comprising a manifold, the manifold comprising a plurality of outlet ports, each outlet port being in fluid communication with one reagent reservoir.

10. The system of claim 1, wherein the liquid reagent comprises oligonucleotide probes.

11. The system of claim 1, wherein the liquid reagent comprises a buffer.

12. The system of claim 11, wherein the liquid reagent comprises a purge fluid, an imaging buffer, or a bleach buffer.

13. A method for using positive pressure in a fluorescent in-situ hybridization imaging system, the method comprising:
applying a positive pressure to a plurality of liquid reagents in a plurality of reagent reservoirs;
selecting a first liquid reagent from the plurality of liquid reagents;
supplying the first liquid reagent as driven by the positive pressure to a flow cell containing a sample; and
obtaining a first fluorescence microscope image of the sample.

14. The method of claim 13, wherein the first liquid reagent comprises oligonucleotide probes having a first nucleotide sequence.

15. The method of claim 14, further comprising
selecting a second liquid reagent from the plurality of liquid reagents; and
supplying the second liquid reagent as driven by the positive pressure to a flow cell containing a sample.

16. The method of claim 15, wherein the second liquid reagent comprises a purge fluid or a photobleaching buffer.

17. The method of claim 14, further comprising
selecting a third liquid reagent from the plurality of liquid reagents;
supplying the third liquid reagent as driven by the positive pressure to a flow cell containing a sample; and
obtaining a second fluorescence microscope image of the sample.

18. The method of claim 17, wherein the third liquid reagent comprises oligonucleotide probes having a third nucleotide sequence.

19. The method of claim 15, wherein the second liquid reagent comprises a purge fluid, a washing bugger, an imaging buffer, or a photobleaching buffer.

20. The method of claim 13, wherein applying the positive pressure comprises applying gas pressure to the plurality of liquid reagents.
